(19) United States
(12) Patent Application Publication
(10) Pub. No.: US 2023/0414889 A1
(43) Pub. Date: Dec. 28, 2023

(54) AUTOMATIC DETECTION OF A DIAPHRAGM IN TIME-MOTION DATA USING VENTILATOR WAVEFORMS

(71) Applicant: KONINKLIJKE PHILIPS N.V., Eindhoven (NL)

(72) Inventors: Jaap Roger Haartsen, Eindhoven (NL); Roberto Buizza, Eindhoven (NL); Cornelis Petrus Hendriks, Eindhoven (NL); Rafael Wiemker, Hamburg (DE); Thomas Koehler, Eindhoven (NL); Joerg Sabczynski, Hamburg (DE); Michael Polkey, London (GB); Nataly Wieberneit, Hamburg (DE)

(21) Appl. No.: 18/206,155

(22) Filed: Jun. 6, 2023

Related U.S. Application Data
(60) Provisional application No. 63/354,818, filed on Jun. 23, 2022.

(51) Int. Cl. A61M 16/00; G16H 20/40; G16H 30/20

(52) U.S. Cl. CPC A61M 16/026; A61M 16/0003; G16H 20/40; G16H 30/20; A61M 2016/0027

(57) ABSTRACT

A diaphragm measurement system (1) includes at least one electronic processor (13) programmed to perform a diaphragm measurement method (100) including receiving ultrasound imaging data (24) of a diaphragm of a patient over a time period encompassing multiple breaths; receiving respiration data of the patient over the time period; calculating a diaphragm thickness metric based on the received ultrasound imaging data of the diaphragm and the received respiration data; and displaying, on a display device (14), a representation (30) of the calculated diaphragm thickness metric.

### CROSS-REFERENCE TO RELATED APPLICATIONS

This patent application claims the priority benefit under 35 U.S.C. § 119(e) of U.S. Provisional Application No. 63/354,818, filed on Jun. 23, 2022, the contents of which are herein incorporated by reference.

The following relates generally to the respiratory therapy arts, mechanical ventilation arts, ventilator induced lung injury (VILI) arts, mechanical ventilation weaning arts, and related arts.

### BACKGROUND

A diaphragm thickening fraction (TFdi or TFDI) as measured by ultrasound (US) is widely recognized to evaluate diaphragm function to optimize ventilator support and weaning. The TFdi is defined as the percentage increase in diaphragm thickness relative to end-expiratory thickness during tidal breathing. The TFdi depends on diaphragmatic activity and reflects the diaphragm work of breathing (WoB) (i.e., the respiratory effort) (see, e.g., Vivier E, Mekontso Dessap A, Dimassi S, et al. “Diaphragm ultrasonography to estimate the work of breathing during non-invasive ventilation.” Intensive Care Med 2012; 38: 796-803; Goligher E C, Fan E, Herridge M S, et al. “Evolution of diaphragm thickness during mechanical ventilation. Impact of inspiratory effort.” Am J Respir Crit Care Med 2015; 192: 1080-1088).

Diaphragm thickness and strain can be assessed at the zone of apposition (ZA) during inspiration and expiration, using a linear high frequency transducer of 10-15 MHz. The zone of apposition is the chest wall area where the lower rib cage reaches the abdominal contents. The probe is positioned between the antero-axillary and mid-axillary lines, perpendicular to the chest wall. With ultrasonic B-mode, the hemi-diaphragm is identified beneath the intercostal muscles as a hypo-echogenic layer of muscle tissue located between two hyper-echogenic lines (the pleural line and the peritoneal line) (see, e.g., Fayssoil A, Behin A, Ogna A, et al. Diaphragm: Pathophysiology and Ultrasound Imaging in Neuromuscular Disorders. J Neuromuscul Di s. 2018). Diaphragmatic thickening is assessed by the thickening fraction (TFdi), calculated as the percentage inspiratory increase in the diaphragm thickness relative to end-expiratory thickness (Tₑₑ) during tidal breathing, i.e., according to Equation 1:

$$TFdi = \frac{{T}_{e i} - {T}_{e e}}{{T}_{e e}} * 100 \% \qquad ( 1 )$$

with Tₑᵢ as the end-inspiratory thickness.

Some studies have evaluated the correlation between TFdi and respiratory effort (see, e.g., M. Umbrello et al. “Diaphragm ultrasound as indicator of respiratory effort in critically ill patients undergoing assisted mechanical ventilation: a pilot clinical study.” Crit Care 19(1): 161, 2015). In this study, the authors found a correlation coefficient of R=0.8 between TFdi and oesophageal pressure-time product and R=0.7 between TFdi and diaphragmatic pressure-time product. In another study (see, e.g., E. Oppersma et al. “Functional assessment of the diaphragm by speckle tracking ultrasound during inspiratory loading.” J Appl Physiology, 123(5):1063-1070, 2017), at the zone of apposition the diaphragm strain can similarly be measured in real-time. For example, in this study, the functional assessment of the diaphragm by speckle tracking ultrasound during inspiratory loading was analyzed. The technique of speckle tracking ultrasound allows for the detection and tracking of diaphragmatic strain over time by analyzing acoustic markers called speckles. These speckles are formed by interference of ultrasound waves that are scattered from physical structures of a size comparable to the wavelength of the ultrasound waves. Both diaphragm strain and diaphragm strain rate were highly correlated to transdiaphragmatic pressure Pdi (strain r²=0.72; strain rate r²=0.80) and EAdi (strain r²=0.60; strain rate r²=0.66).

Moreover, the use of ultrasound to evaluate the respiratory muscle function (especially the diaphragm) is relatively new and remains infrequent due to the supposed difficulty in obtaining adequate measurements (see, e.g., Aarab Y, Jaber S, De Jong A, “Diaphragm Ultrasonography in ICU: Why, How, and When To Use It?” ICU Management & Practice, Volume 21—Issue 3, 2021; Tuinman, P. R., Jonkman, A. H., Dres, M. et al. “Respiratory muscle ultrasonography: methodology, basic and advanced principles and clinical applications in ICU and ED patients—a narrative review.” Intensive Care Med 46, 594-605 (2020)). Possible confounders reducing the reproducibility and accuracy of, for example, daily bed-side measurements are varying conditions such as a location of the US-probe at each measurement on the patient, an attitude (angulation) of the probe, a phase point in the patient's respiratory cycle, a manually exerted skin pressure of the probe, a user-chosen ultrasound settings, a thickening fraction varying over the extent of the diaphragm muscle, an effort to record, store, and process the data points manually, and so forth.

Diaphragm thickness and thickness fraction can be assessed using B-mode and M-mode imaging (see, e.g., Kahn B S, Gursel G. “Does it make difference to measure diaphragm function with M mode (MM) or B mode (BM)?” J Clin Monit Comput. Vol. 34, 1247-1257, 2020). In case of M-mode first a 2D B-mode movie is recorded. From the images a single scan line is selected that intersects the diaphragm region of interest. Next, a time-motion image of that scan line is plotted from which the diaphragm thickness and thickness fraction can be determined.

Wearable ultrasound patches for non-invasive continuous assessment of diaphragm thickness can be used in several potential applications such as continuous atrophy detection, diaphragm dysfunction detection, accurate breathing rate detection, weaning prediction, asynchrony detection, and proportional ventilation (noninvasive NAVA).

Ideally, an ultrasound patch to measure the thickness (or thickening fraction) of the diaphragm is designed to be operated in M-mode, i.e. to make one or a few scan lines instead of a full two-dimensional (2D) image. This, to reduce cost, footprint, power consumption, and amount of data that needs to be processed and transferred. However, without a full 2D image it is difficult to identify the diaphragm in a single scan line.

The following discloses certain improvements to overcome these problems and others.

### SUMMARY

In one aspect, a diaphragm measurement system includes at least one electronic processor programmed to perform a diaphragm measurement method including receiving ultrasound imaging data of a diaphragm of a patient over a time period encompassing multiple breaths; receiving respiration data of the patient over the time period; calculating a diaphragm thickness metric based on the received ultrasound imaging data of the diaphragm and the received respiration data; and displaying, on a display device, a representation of the calculated diaphragm thickness metric.

In another aspect, a diaphragm measurement method includes, with at least one electronic processor, receiving ultrasound imaging data of a diaphragm of a patient over a time period encompassing multiple breaths; receiving respiration data of the patient over the time period; calculating a diaphragm thickness metric based on the received ultrasound imaging data of the diaphragm and the received respiration data; and displaying, on a display device, a representation of the calculated diaphragm thickness metric.

One advantage resides in using a wearable ultrasound (US) patch to acquire US imaging data of a patient.

Another advantage resides in determining a location of a diaphragm of a patient from US imaging data.

Another advantage resides in determining a location of a diaphragm of a patient without a full US image.

Another advantage resides in controlling settings of a US patch to acquire US imaging data of a patient.

Another advantage resides in controlling settings of a mechanical ventilator based on US imaging data of a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation system in accordance with the present disclosure.

FIG. 2 shows an example flow chart of operations suitably performed by the system of FIG. 1.

### DETAILED DESCRIPTION

As used herein, the singular form of “a”, “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

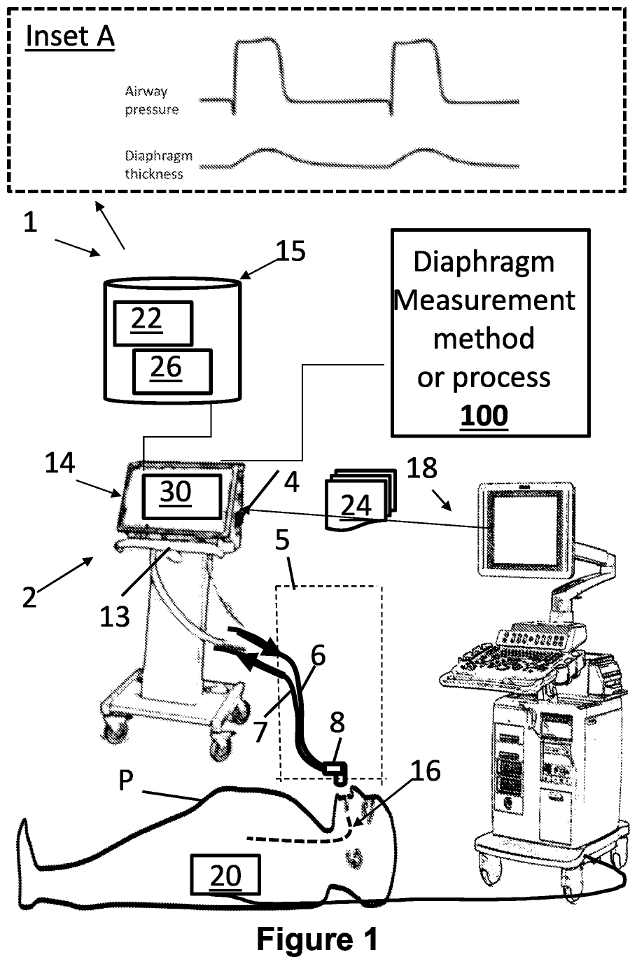

With reference to FIG. 1, a diaphragm measurement device or system 1 is shown. A mechanical ventilator 2 is configured to provide ventilation therapy to an associated patient P is shown. As shown in FIG. 1, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 shows the patient P already intubated. That is, FIG. 1 shows the patient after a tracheal intubation has been performed to insert the ETT 16 into the patient. However, to safely perform the tracheal intubation, the anesthesiologist or other qualified medical professional first performs an assessment of the patient P to select the ETT size of the ETT 16, and then inserts an ETT of the selected size into the patient P by a tracheal intubation procedure.

FIG. 1 also shows a medical imaging device 18 (also referred to as an image acquisition device, imaging device, and so forth). As primarily described herein, the medical imaging device 18 comprises an ultrasound (US) medical imaging device 18. In other embodiments, the image acquisition device 18 can be a Computed Tomography (CT) image acquisition device, a C-arm imager, or other X-ray imaging device; Magnetic Resonance (MR) image acquisition device; or a medical imaging device of another modality. As described herein, the medical imaging device 18 is used to acquire images of the patient P. In some embodiments, the medical imaging device 18 can comprise a wearable US imaging device 18. In other embodiments, the medical imaging device 18 can comprise a US imaging probe 18.

In a more particular example, the medical imaging device 18 includes an ultrasound patch 20 that is wearable by the patient P (e.g., on the abdomen or chest of the patient P in position to image the diaphragm of the patient, as shown in FIG. 1). The US patch 20 comprises one or more transducers, and is positioned to acquire US imaging data (i.e., US images) 24 of the diaphragm of the patient P. For example, the US patch 20 is configured to acquire imaging data of a diaphragm of the patient P, and more particularly US imaging data related to a thickness of the diaphragm of a patient P during inspiration and expiration while the patient P undergoes mechanical ventilation therapy with the mechanical ventilator 2. The electronic processor 13 controls the ultrasound imaging device 18 to receive the ultrasound imaging data 24 of the diaphragm of the patient P from the handheld US patch 20. In other embodiments, the electronic processor 13 can be implemented in the ultrasound imaging device 18 and can be configured to control operation of both the ultrasound imaging device 18 and/or the mechanical ventilator 2. In further embodiments, the electronic processor 13 can be implemented in a separate electronic processing device (not shown), for example a computer, a smart tablet, and so forth.

In some embodiments, the non-transitory computer readable medium 15 stores an artificial neural network (ANN) model 22 configured to determine a diaphragm thickness metric based on the ultrasound imaging data 24 and respiration data of the patient. The non-transitory computer readable medium 15 also stores instructions executable by the electronic controller 13 to perform a diaphragm measurement method or process 100.

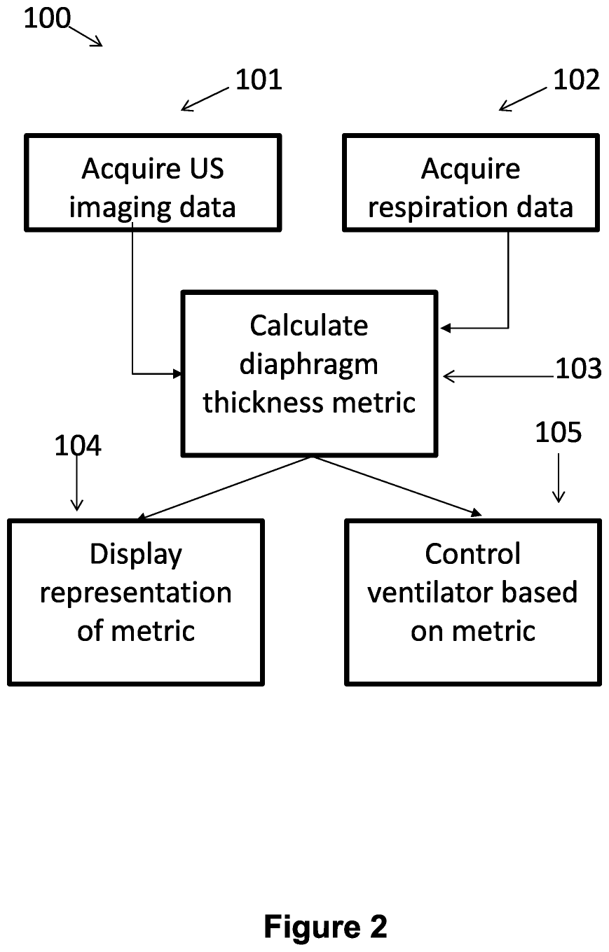

With reference to FIG. 2, and with continuing reference to FIG. 1, an illustrative embodiment of the diaphragm measurement method 100 is diagrammatically shown as a flowchart. At an operation 101, the US imaging data 24 of the diaphragm of the patient P is received over a time period encompassing multiple breaths. For example, the electronic processor 13 controls the ultrasound imaging patch 20 to obtain the ultrasound imaging data 24 of the diaphragm of the patient P.

At an operation 102, respiration data of the patient is received over the time period. The respiration data can be, for example, airway pressure (from a pressure sensor), airway flow (from an air flow sensor), or an output of a respiration monitor (such as a respiration belt worn by the patient P).

At an operation 103, a diaphragm thickness metric can be calculated based on the received US imaging data 24 of the diaphragm of the patient P and the received respiration data. In one example, the diaphragm thickness metric includes a diaphragm thickening ratio indicative of a diaphragm thickness during inspiration relative to a diaphragm thickness during expiration. In another example, the diaphragm thickness metric includes a mean diaphragm thickness over multiple respiratory cycles.

At an operation 104, a representation 30 of the calculated diaphragm thickness metric is displayed on the display device 14 of the mechanical ventilator 2. In some embodiments, at an operation 105, the mechanical ventilator 2 can be controlled to adjust one or more parameters of the mechanical ventilation therapy delivered to the patient based on the calculated diaphragm thickness metric.

In some embodiments, the US imaging data 24 comprises M-mode US imaging data. For example, the diaphragm can be automatically detected in a single channel ultrasound echo pattern, thereby making use of the correlation between airway pressure and diaphragm thickness of actively breathing patients receiving pressure support ventilation, or pressure or volume controlled ventilation. For example, for pressure support ventilation mode airway pressure correlates with diaphragm thickness, as shown in Inset A of FIG. 1. In such embodiments, the calculating of the diaphragm thickness metric based on the received M-mode ultrasound imaging data 24 of the diaphragm and the received respiration data includes identifying a component of the M-mode ultrasound imaging data 24 corresponding to the diaphragm of the patient P based on the respiration data of the patient P, and calculating the diaphragm thickness metric based on the identified component of the M-mode ultrasound imaging data 24 corresponding to the diaphragm of the patient P. To do so, hyper-echogenic lines in the M-mode ultrasound imaging data 24 are identified, and grouped into pair of hyper-echogenic lines. For example, the hyper-echogenic lines can be identified by thresholding, edge-detection, or any other suitable technique. Alternatively, this is done on the raw ultrasound data 24 before it is converted into a grayscale image by identifying the high amplitude parts in the echo pattern (i.e. the strong echoes reflected from hyper-echogenic structures). The pairs of the hyper-echogenic lines represent possible boundaries of the diaphragm. Preferably, adjacent lines are grouped together since the diaphragm should appear as a hypo-echogenic layer of muscle tissue located between two adjacent hyper-echogenic lines, namely the pleural line and the peritoneal line.

For each pair of hyper-echogenic lines, a distance between the hyper-echogenic lines of the pair is determined as a function of time, and a correlation between the determined distance between the hyper-echogenic lines of the pair as a function of time and the respiration data of the patient P is determined. The component of the M-mode ultrasound imaging data 24 corresponding to a diaphragm of the patient P is identified as one of the pairs of hyper-echogenic lines based on the determined correlations. Since for pressure support ventilation airway pressure correlates with diaphragm thickening, the pair with the largest correlation coefficient is selected as being the two hyper-echoic lines in between which the diaphragm is located. For example, a respiration rate of the patient over the time period is identified from the respiration data of the patient P, and the M-mode ultrasound imaging data 24 is filtered using a bandpass filter 26 (for example, implemented in the non-transitory computer readable medium 15 of the mechanical ventilator 2) with a passband centered at the identified respiration rate to extract the component of the M-mode ultrasound imaging data 24 corresponding to the diaphragm of the patient P.

In the case of multiple scan lines, the most optimal scan line could be selected by selecting the scan line with the largest correlation coefficient. Also, multiple scan lines could be selected (e.g., for those correlation coefficients exceeding a pre-determined threshold level) and a mean or median diaphragm thickness could be calculated. If the correlation coefficient(s) do not exceed a pre-determined threshold level the user/caregiver may be warned (e.g., to reposition the patch 20 or to check for partial or complete (paralyzed) diaphragm dysfunction). The procedure could be repeated on a regular (e.g., daily) basis.

In some embodiments, the US imaging data 24 of the diaphragm of the patient P is received over a time period encompassing multiple breaths, for example, during inspiration and expiration while the patient P undergoes mechanical ventilation therapy with the mechanical ventilator 2. In such embodiments, the electronic controller 13 is configured to determine a phase shift between the bandpass-filtered M-mode ultrasound imaging data 24 and the respiration data of the patient P. A patient-ventilator asynchrony is determined based on the phase shift, and an indication of the determined patient-ventilator asynchrony is displayed on the display device 14 of the mechanical ventilator 2. To do so, the US imaging data 24 and the respiration data are acquired, and a frequency of the mechanical ventilator 2 is determined. The US imaging time motion data is filtered, and the phase shift between the signal of the muscle thickening from the US time motion data and the ventilator pressure signal is determined.

In some embodiments, the diaphragm measurement method 100 can be repeated for successive sessions based on adjustments to the system 1. In one example, one or more settings of the mechanical ventilator 2 are adjusted, and the method 100 is repeated for each adjustment. For example, the diaphragm is identified by changing ventilator settings and making use of the negative correlation between the diaphragm thickening fraction and a pressure support level actively breathing patients receiving pressure support to identify the diaphragm in the echo pattern. To do so, a ventilator support level is set. Hyper-echogenic lines in the time motion data are identified, and a distance between each pair of adjacent high amplitude parts is determined. For each breath, the time stamps of end-of-inspiration and end-of-expiration are determined from the pressure or flow waveforms. For each pair, the thickness fraction is calculated using the time stamps. These operations are repeated for several support levels. For each pair, the correlation coefficient between support level and maximum thickness is determined. The pair with largest correlation coefficient is selected as being the two hyper-echogenic lines between which the diaphragm is located.

In the case of multiple scan lines, the optimal scan line could be selected by repeating these operations for each scan line and selecting the scan line with the largest correlation coefficient. Also, multiple scan lines could be selected e.g. for those correlation coefficients exceeding a pre-determined threshold level, and a mean or median diaphragm thickness could be calculated. In addition, if the correlation coefficient(s) don't exceed a pre-determined threshold level the user/caregiver may be warned e.g. to reposition the patch 20.

In another example, one or more settings of the ultrasound patch 20 (such as frequency, time-variable gain, steering angle, focus depth, and aperture size and position (i.e. selection of active elements in the ultrasound array) are adjusted, and the method 100 is repeated for each adjustment. The ultrasound patch 20 is equipped with a transducer array that allows for steering, several scan lines could be recorded by sweeping the angle. The optimal scan line could be selected, or an average thickness (fraction) could be calculated. Similarly, optimal settings could be found for frequency, focus depth, etc.

In another embodiment, the calculating of the diaphragm thickness metric can include inputting the M-mode ultrasound imaging data 24 and the respiration data to the ANN model 22 to determine the diaphragm thickness metric based on the M-mode ultrasound imaging data 24 and the respiration data of the patient P. The correlation between pressure and diaphragm thickness is learned implicitly during training of the ANN model 22. To do so, the M-mode ultrasound imaging data 24 and the respiration data are acquired and input to the ANN model 22, and the ANN model 22 is trained to reproduce the ground truth diaphragm.

In some embodiments, the mechanical ventilator (MV) 2 provides several types of waveforms (e.g. pressure, flow, work-of-breathing, volume, etc). For each mutual pair of the US-line and the MV-signal, the correlation is determined, and the optimally correlating selection of the MV-waveform is determined. In cases of partial or complete diaphragm dysfunction, under pressure or volume-controlled ventilation, the diaphragm thickness fraction can be negative due to passive stretching (see, e.g., Santana P V, Cardenas L Z, Albuquerque A L P, Carvalho C R R, Caruso P. Diaphragmatic ultrasound: a review of its methodological aspects and clinical uses. J Bras Pneumol. 2020 Nov. 20). Also, other factors can lead to a negative diaphragm thickness fraction, such as a too high-pressure support level. Hence, when detecting a negative diaphragm thickness fraction, a warning could be given to the caregiver or ventilation settings could automatically be adapted.

While described in terms of mechanical ventilation therapy, the system 1 can be used in any suitable setting, including heart surgery, heart failure monitoring, asynchrony detection, home ventilation, bladder and prostate enlargement monitoring, carotid artery narrowing (e.g., stroke, cardiac output, A-fib, and so forth), heart muscle function and cardiac output in an acute care setting (i.e., heart attack), and so forth.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A diaphragm measurement system, comprising:
at least one electronic processor programmed to perform a diaphragm measurement method including:
receiving ultrasound imaging data of a diaphragm of a patient over a time period encompassing multiple breaths;
receiving respiration data of the patient over the time period;
calculating a diaphragm thickness metric based on the received ultrasound imaging data of the diaphragm and the received respiration data; and
displaying, on a display device, a representation of the calculated diaphragm thickness metric.

2. The system of claim 1, wherein the diaphragm thickness metric includes a diaphragm thickening ratio indicative of a diaphragm thickness during inspiration relative to a diaphragm thickness during expiration.

3. The system of claim 1, wherein the diaphragm thickness metric includes a mean diaphragm thickness over multiple respiratory cycles.

4. The system of claim 1, further comprising:
an ultrasound imaging patch wearable by the patient, wherein the at least one electronic processor controls the ultrasound imaging patch to obtain the ultrasound imaging data of the diaphragm of the patient.

5. The system of claim 1, wherein the ultrasound imaging data comprises M-mode ultrasound imaging data, and the calculating of the diaphragm thickness metric based on the received M-mode ultrasound imaging data of the diaphragm and the received respiration data includes:
identifying a component of the M-mode ultrasound imaging data corresponding to the diaphragm of the patient based on the respiration data of the patient; and
calculating the diaphragm thickness metric based on the identified component of the M-mode ultrasound imaging data corresponding to the diaphragm of the patient.

6. The system of claim 5, wherein the at least one electronic processor is programmed to identify the component of the M-mode ultrasound imaging data corresponding to the diaphragm of the patient by:
identifying hyper-echogenic lines in the M-mode ultrasound imaging data;
grouping the hyper-echogenic lines into pairs of hyper-echogenic lines;
for each pair of hyper-echogenic lines, determining a distance between the hyper-echogenic lines of the pair as a function of time;
for each pair of hyper-echogenic lines, determining a correlation between the determined distance between the hyper-echogenic lines of the pair as a function of time and the respiration data of the patient; and
identifying the component of the M-mode ultrasound imaging data corresponding to a diaphragm of the patient as one of the pairs of hyper-echogenic lines based on the determined correlations.

7. The system of claim 5, wherein identifying the component of the M-mode ultrasound imaging data corresponding to the diaphragm of the patient based on the respiration data of the patient includes:
identifying a respiration rate of the patient over the time period from the respiration data of the patient; and
filtering the M-mode ultrasound imaging data using a bandpass filter with a passband centered at the identified respiration rate to extract the component of the M-mode ultrasound imaging data corresponding to the diaphragm of the patient.

8. The system of claim 7, wherein the patient is receiving mechanical ventilation over the time period, and the electronic processor is further programmed to:
determine a phase shift between the bandpass-filtered M-mode ultrasound imaging data and the respiration data of the patient;
determine a patient-ventilator asynchrony based on the phase shift; and
display, on the display device, an indication of the determined patient-ventilator asynchrony.

9. The system of claim 1, wherein the respiration data of the patient comprises airway pressure, airway flow, or an output of a respiration monitor.

10. The system of claim 1, wherein the patient is receiving mechanical ventilation over the time period, and the electronic processor is programmed to:
adjust one or more settings of a mechanical ventilator and repeat the diaphragm measurement method for each adjustment.

11. The system of claim 1, wherein the electronic processor is programmed to:
adjust one or more settings of an ultrasound patch configured to acquire the ultrasound imaging data and repeat the diaphragm measurement method for each adjustment.

12. The system of claim 1, wherein the calculating of the diaphragm thickness metric based on the received ultrasound imaging data of the diaphragm and the received respiration data includes
inputting the ultrasound imaging data and the respiration data to an artificial neural network (ANN) model configured to determine the diaphragm thickness metric based on the ultrasound imaging data and the respiration data of the patient.

13. The system of claim 1, wherein the at least one electronic processor is configured to:
control an associated mechanical ventilator to adjust one or more parameters of mechanical ventilation therapy delivered to the patient based on the calculated diaphragm thickness metric.

14. The system of claim 13, further comprising:
a mechanical ventilator configured to deliver mechanical ventilation therapy to the patient.

15. A diaphragm measurement method comprising, with at least one electronic processor:
receiving ultrasound imaging data of a diaphragm of a patient over a time period encompassing multiple breaths;
receiving respiration data of the patient over the time period;
calculating a diaphragm thickness metric based on the received ultrasound imaging data of the diaphragm and the received respiration data; and
displaying, on a display device, a representation of the calculated diaphragm thickness metric.
